(19) United States
(12) Patent Application Publication
(10) Pub. No.: US 2011/0308533 A1
(43) Pub. Date: Dec. 22, 2011

(54) APPARATUS AND METHOD OF ANCHORING AN EAR PIECE

(76) Inventor: Devin Benner, Pleasant Ridge, MI (US)

(21) Appl. No.: 13/127,500

(22) PCT Filed: Nov. 5, 2009
(86) PCT No.: PCT/US09/63411

§ 371 Date: Sep. 8, 2011

Related U.S. Application Data
(60) Provisional application No. 61/111,447, filed on Nov. 5, 2008.

(51) Int. Cl. A61F 11/10

(52) U.S. Cl. 128/865

(57) ABSTRACT

An apparatus and method of anchoring an earpiece is provided. The apparatus includes a flexible member of moldable expandable, semi-adhesive material. The flexible member has a predetermined shape. The method of anchoring an earpiece includes the steps of placing an earpiece in the ear. The method also includes the steps of shaping the flexible member into a ball and placed the shaped flexible member into the concha portion of the ear, such that the shaped flexible member is disposed the earpiece and the antihelix and the earpiece anchor expands to retain the earpiece in the ear.

### CROSS REFERENCE TO RELATED APPLICATIONS

This application claims the benefit of and priority to U.S. Provisional Patent Application No. 61/111,447, filed Nov. 5, 2008 which is incorporated by reference herein in its entirety.

### BACKGROUND

1. Field

The present invention relates to an earpiece, and in particular to an apparatus and method of anchoring an earpiece positioned within an ear.

2. Description of the Related Art

Currently, various types of earpieces are placed within a user's ear. For example, the earpiece may be an ear plug that blocks the transmission of sound into the ear. In another example, the ear piece may be a listening device. Examples of listening devices include earphones, headphones, ear buds, hearing aids or the like. At the same time, human ears come in various shapes and sizes, and it may be difficult to retain the ear piece within the user's ear.

Thus, there is a need in the art for a multifunctional earpiece or anchor that can accommodate various types of earpieces, as well as user ear shapes and sizes and yet is retained within the user's ear regardless of the activity of the user.

### SUMMARY

An apparatus and method of anchoring an earpiece is provided. The apparatus includes a flexible member of moldable, expandable, semi-adhesive material. The flexible member has a predetermined shape.

Also provided, a method of anchoring an earpiece includes the steps of placing an earpiece in the ear. The method also includes the steps of shaping the flexible member into a ball and placed the shaped flexible member into the concha portion of the ear, such that the shaped flexible member is disposed between the earpiece and the antihelix and the earpiece anchor expands to retain the earpiece in the ear.

An advantage of the present apparatus and method is that it is customizable to the user. Another advantage is that the apparatus and method is adaptable to various styles of earpieces. A further advantage is that the apparatus and method holds the earpiece without blocking the ear canal. Still a further advantage is that the apparatus and method preserves the sound quality of the earpiece. Yet a further advantage is that the apparatus is cost effective to produce

Other features and advantages of the present disclosure will be readily appreciated, as the same becomes better understood after reading the subsequent description taken in conjunction with the accompanying drawings.

### BRIEF DESCRIPTION OF THE DRAWINGS

FIG. 1 is an elevational view of an earpiece anchor prior to installation.

FIG. 2 is an elevational view of an ear with the earpiece anchor of FIG. 1 positioned therein.

FIG. 3a is a diagram illustrating a method of anchoring an earpiece using the earpiece anchor of FIG. 1.

FIG. 3b is a side view of an earpiece retained within an ear by the earpiece anchor of FIG. 1.

### DETAILED DESCRIPTION

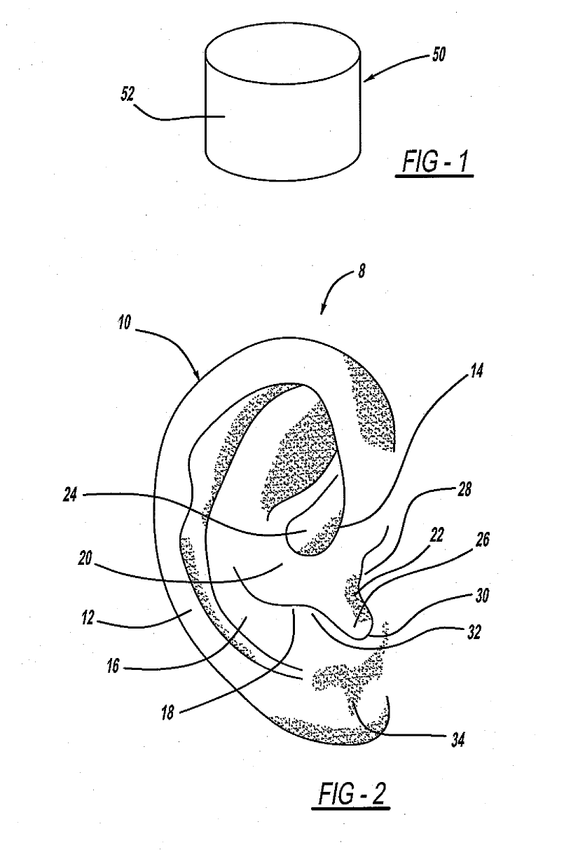

Referring to FIG. 1 and FIG. 2, an earpiece anchor 50 is illustrated for use in an ear 8. The ear 8 includes an external portion or outer ear 10. The outer ear 10 includes a flap of cartilage referred to as the pinna and the ear canal.

The outer ear 10 or pinna has an exterior rim or helix 12 that begins at an upper base region of the pinna referred to as a crux of helix 14, and extends around the outer edge of the pinna into the ear lobe 34. An interior portion of the pinna includes a y-shaped elevated ridge referred to as an antihelix 16. The antihelix 16 extends from an upper base region 18 and defines a boundary of a cavity referred to as a concha 20. The concha 20 leads into the ear canal 22 and includes an upper concha 24 and a lower concha 26. The ear also includes a small notch in front of the auditory canal referred to as a tragus 28, an intertragal notch 30, located at the lowermost portion of the concha bowl and an anti tragus 32, which is a ridge opposite the targus.

The lower concha, intertragal notch and antitragus define a wedge-shaped space having an apex that generally points towards the chin of the individual and leads into the ear canal. The earpiece is positioned within this wedge shaped space defined by the concha as shown at 62.

The earpiece anchor 50 initially has a predetermined shape. The initial predetermined shape may be spherical, or cubical, or cylindrical or the like. The earpiece anchor 50 may have a diameter of about ½ in. The earpiece anchor 50 is made from a flexible material that can be molded or shaped. Preferably, the material used to make the earpiece anchor 50 is suitable for human use. An example of such a material is a foam such as a urethane, polyvinylchloride or the like that can be compressed, and then expands and conforms to the shape of the environment. Another example of a material is a silicone putty that relies on the application of pressure to shape. In addition, an outer surface 52 of the earpiece anchor 50 has an adhesive quality to assist in retaining the anchor 50 in the ear 8, and to prevent movement of the earpiece anchor 50. An outer surface 52 of the material may provide a semi-adhesive, adhesive, or tacky property to the earpiece anchor 50, to allow the ear piece anchor to partially adhere to the ear or earpiece and/or resist sliding or movement or the earpiece anchor. The semi-adhesive property can be intrinsic to the material used for the earpiece anchor, or be extrinsic to the material. For example and an extrinsic adhesive coating (e.g., silicone or latex based adhesives, tape, and the like) can be applied to the outer surface of the earpiece anchor, or the like. An example of a material with an intrinsic adhesive property is silicone putty, urethane foam, polyvinylchloride foam, and the like. The adhesive is preferably a weak, non-permanent adhesive that is approved for human use.

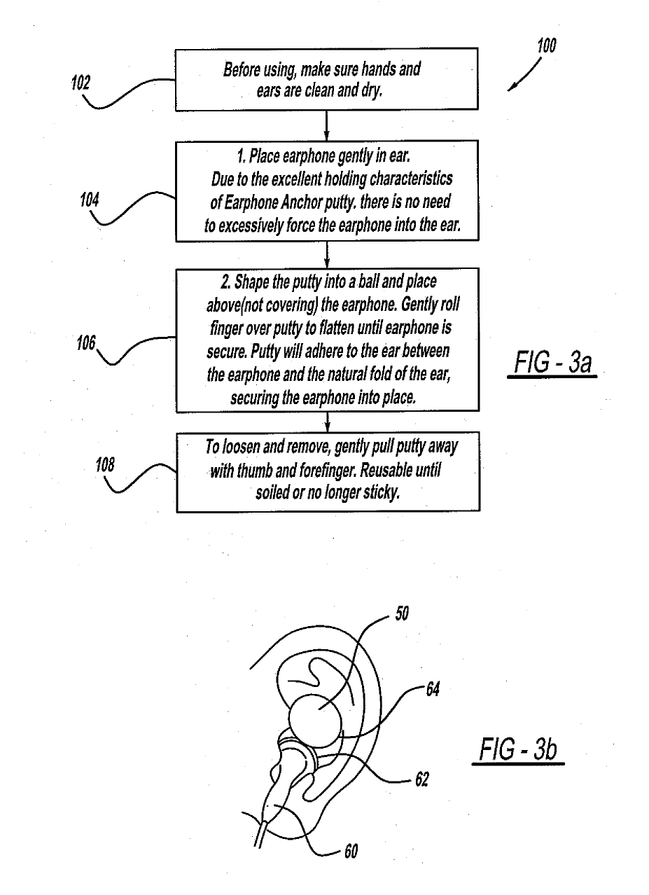

In operation, the method 100 of using the earpiece anchor 50 to retain an earpiece 60 includes a first step 102 of placing the earpiece 60 into a predetermined position with respect to the ear 8, as shown in FIG. 3a. Generally, the earpiece is positioned within the lower concha cavity as shown at 62. In a second step 104, the earpiece anchor 50 is compressed, such as by shaping the earpiece anchor 50 into a predetermined shape, such as a sphere or ball. In a third step 106, the earpiece anchor 50 is positioned within the concha, such that it is disposed between the earpiece and the antihelix as shown at 64. In a fourth step 108, the user secures the earpiece 60 using the earpiece anchor 50. For example, the user's finger rolls across the exposed surface of the earpiece anchor 50 and the earpiece anchor 50 expands and generally conforms to the shape of the concha cavity, as shown in FIG. 3b. The adhesive outer surface 52 of the earpiece anchor 50 assists in the retention of the earpiece anchor 50 and the earpiece 60. The earpiece anchor 50 is removable by the user pulling the earpiece anchor 50 in an outwardly direction away from the ear 8. It should be appreciated that the earpiece anchor 50 can be reshaped, and is reusable.

The present invention has been described in an illustrative manner. It is to be understood that the terminology which has been used is intended to be in the nature of words of description rather than of limitation.

Many modifications and variations of the present invention are possible in light of the above teachings. Therefore, the present invention may be practiced other than as specifically described.

What is claimed is:

1. An apparatus for anchoring an earpiece within an outer ear of the user comprising:
an earpiece anchor that is moldable and expandable, wherein the earpiece anchor has a predetermined initial shape for retaining the earpiece within the outer ear of the user.

2. The apparatus for anchoring an earpiece within an outer ear of the user of claim 1, wherein the anchor member is made from a flexible material that may be compressed and thereafter expanded to conform to the shape of the outer ear of the user to retain the earpiece.

3. The apparatus for anchoring an earpiece within an outer ear of the user of claim 2, wherein the flexible material is one of urethane and polyvinylchloride.

4. The apparatus for anchoring an ear piece within an outer ear of the user of claim 1, wherein the anchor member is made from a moldable material that may be shaped to conform to the contoured surfaces and irregular shape of the outer ear and positioned between the earpiece and a portion of the outer ear.

5. The apparatus for anchoring an ear piece within an outer ear of the user of claim 4, wherein the moldable material is a silicone putty.

6. The apparatus for anchoring an ear piece within an outer ear of the user of claim 1, wherein the anchor member is made from a material having an outer surface, the outer surface having one of a semi-adhesive, adhesive, and tacky property.

7. The apparatus for anchoring an earpiece within an outer ear of the user of claim 6, wherein the property is intrinsic to the material.

8. The apparatus for anchoring an earpiece within an outer ear of the user of claim 6, wherein the outer surface of the earpiece anchor has one of a semi-adhesive, adhesive, and tacky coating.

9. The apparatus for anchoring an earpiece within an outer ear of the user of claim 8, wherein the earpiece anchor is re-usable and can be reshaped.

10. A method of retaining an earpiece in an ear, said method comprising the steps of:
placing an earpiece in an ear of a user, wherein the earpiece is positioned in a concha cavity found in an outer ear of the user;
compressing the earpiece anchor, wherein the earpiece anchor is a moldable, expandable member having a predetermined initial shape;
placing the earpiece anchor into the ear, such that the earpiece anchor is disposed between the ear piece and an antihelix portion of the outer ear, and
retaining the earpiece in the outer ear by applying pressure to the earpiece anchor to flatten the earpiece anchor to a second retained shape.

11. The method of retaining an earpiece in an ear of claim 10, further comprising the step of:
removing the earpiece anchor from the ear; and reshaping and reusing the earpiece anchor at a later time.
